(19) United States
(12) Patent Application Publication
(10) Pub. No.: US 2021/0113842 A1
(43) Pub. Date: Apr. 22, 2021

(54) PACE DETECTION IN ECG WAVEFORMS

(71) Applicant: Analog Devices, Inc., Norwood, MA (US)

(72) Inventor: Shrenik DELIWALA, Andover, MA (US)

(73) Assignee: Analog Devices, Inc., Norwood, MA (US)

(21) Appl. No.: 16/987,743

(22) Filed: Aug. 7, 2020

Related U.S. Application Data
(60) Provisional application No. 62/924,237, filed on Oct. 22, 2019.

(51) Int. Cl. A61N 1/37

(52) U.S. Cl. CPC A61N 1/371; A61N 1/3704

(57) ABSTRACT

System and apparatus for pace detection and implementation thereof. A device for detecting pace pulse in the presence of ECG waveforms is disclosed. Specifically, the present disclosure relates to an elegant, novel circuit and method for presenting pace pulse data with or without ECG signals measuring for use in useful in a variety of medical applications. This allows for robust, portable, low-power, higher S/N devices which have historically required a much bigger footprint and complexity.

### CROSS-REFERENCE TO RELATED APPLICATIONS

This application is related to and claims the benefit of priority under 35 U.S.C. § 119(e) to U.S. Provisional Patent Application No. 62/924,237 entitled, “PACE DETECTION IN ECG WAVEFORMS” filed on Oct. 22, 2019 and related to U.S. Provisional Application No. 62/511,709 entitled, “Measuring Bio-Potentials” filed on May 26, 2017, and U.S. patent application Ser. No. 15/988,378 entitled, “BIOPOTENTIAL MEASUREMENT SYSTEM AND APPARATUS” filed on May 24, 2018, all of which are hereby incorporated by reference in their entirety.

### FIELD OF THE DISCLOSURE

The present disclosure relates to medical equipment. More specifically, this disclosure describes apparatuses and systems for pace detection while measuring biopotentials.

### BACKGROUND

Biopotential measurement is can be used in modern medical procedures. For example, biopotentials can be used for electrocardiogram (ECG), electroencephalogram (EEG), electromyography (EMG), etc. ECG lead systems are used to obtain biopotential signals containing information indicative of the electrical activity associated with the heart and pulmonary system.

Correct detection of pacemaker pulses in the electrocardiogram (ECG) is crucial for proper evaluation of the effect of a pacemaker on the cardiac rhythm. The ECG signal recorded from a patient with implanted cardiac pacemaker consists of three parts: the natural ECG signal, pacemaker pulses and noise. The informative content of the ECG signal lies in the frequency band (0-150) Hz and its dynamic range is usually up to 2 mV. On the other hand, the pacing pulses have a typical duration from 0.1 ms to 2 ms and amplitude higher than 0.5 mV.

Pacing pulses have very fast rising and falling edges—the rising edge duration could be 100 ns measured at the pacemaker leads, appearing widened to 10 μs on the surface of the human body. The detection of pacing and pacing artifacts is important, since they indicate the presence of a pacemaker and help to evaluate the reaction of the heart.

There are different medical standards with variable requirements regarding the height and width of the pace pulse that has to be captured and indicated on the screen of the device. The desired features of the pacemaker pulses that should be detected comprise: duration—0.1 ms to 2 ms; amplitude—2 mV to 250 mV; frequency—up to 100 impulses per minute; and, rising edge duration—less than 100 ms.

Many techniques have been proposed to tease out pace pulses from ECG signals. Unfortunately, their sensor complexity, computational demands and power consumption makes many of them impermissible for the vast majority of professional uses, as well as a large proportion portable which is a burgeoning field. The inventor of the present disclosure has identified these shortcomings and recognized a need for a more elegant, robust pace detection system with a small footprint. That is, a pace detector which is simple enough for ubiquitous use while being versatile for portable devices.

This overview is intended to provide an overview of subject matter of the present patent application. It is not intended to provide an exclusive or exhaustive explanation of the invention. Further limitations and disadvantages of conventional and traditional approaches will become apparent to one of skill in the art, through comparison of such systems with some aspects of the present invention as set forth in the remainder of the present application with reference to the drawings.

### SUMMARY OF THE DISCLOSURE

System and apparatus for pace detection and implementation thereof. A device for detecting pace pulse in the presence of ECG waveforms is disclosed. Specifically, the present disclosure relates to an elegant, novel circuit and method for presenting pace pulse data with or without ECG signals measuring for use in useful in a variety of medical applications. This allows for robust, portable, low-power, higher S/N devices which have historically required a much bigger footprint and complexity.

According to one aspect, the present disclosure is an apparatus for measuring pace pulses using a sense capacity and altering the sampling rate accordingly.

According to another aspect of the disclosure, pace detector sampling rate is maximized by calculating the RC time constant of the medical apparatus such that the sampling rate is low enough to ensure that the sense capacity discharges before the next sample yet fast enough to yield a sample window which captures the pace pulse event.

According to another aspect of the disclosure, the sample is then filtered.

According to another aspect of the disclosure, the filter is a low pass filter.

According to another aspect of the disclosure, the filter is a discreet time filter.

According to another aspect of the disclosure, the filter is a low-pass filter.

According to another aspect of the disclosure, the filter is a median filter.

According to another aspect of the disclosure, pace detector analyzes the filtered data by looking for a spike.

According to another aspect of the disclosure, the spike analysis is performed in the time domain.

According to another aspect of the disclosure, the spike analysis is performed in the frequency domain.

According to another aspect of the disclosure, the spike analysis uses a threshold to identify the pulse.

According to another aspect of the disclosure, the threshold is set to 3 sigmas of the noise present in the filter.

According to another aspect of the disclosure, the thresholding of 3 sigma is equivalent to a bit error rate (BER) of 10-3.

According to another aspect of the disclosure, pace detector comprises a first electrode electrically connected in series to a first high resistance resistor.

According to another aspect of the disclosure, pace detector comprises a second electrode electrically connected in series to a second high resistance resistor.

According to another aspect of the disclosure, pace detector further comprises a first electrode electrically connected in series to a first high resistance resistor.

According to another aspect of the disclosure, pace detector further comprises an amplifier electrically connected to the first high resistance resistor.

According to another aspect of the disclosure, pace detector wherein the amplifier electrically connected to the second high resistance resistor.

According to another aspect of the disclosure, pace detector wherein the amplifier is electrically connected to the first high resistance resistor through a switch.

According to another aspect of the disclosure, pace detector wherein, the pace detector is configured to explicitly measure the currents flowing in an external circuit, in which the currents are restricted to a small value by a large series resistor (Rs) by design.

According to another aspect of the disclosure, pace detector further comprises a capacitor electrically connected in parallel between the resistor and the amplifier.

According to another aspect of the disclosure, pace detector wherein, the measurement is configured by average current in a given time interval by charging a capacitor via a large resistor.

According to another aspect of the disclosure, pace detector wherein, the measurement is configured for sequential read-out of multiple potential using the same amplifier.

According to yet another aspect of the disclosure, pace detector wherein, the measurement of electrode impedance is performed by measuring currents thru the same amplifier used for measuring biopotential generated currents.

The drawings show exemplary biopotential and pace detection circuits and configurations. Variations of these circuits, for example, changing the positions of, adding, or removing certain elements from the circuits are not beyond the scope of the present invention. The illustrated pace detectors, configurations, and complementary devices are intended to be complementary to the support found in the detailed description.

### BRIEF DESCRIPTION OF THE DRAWINGS

The present disclosure is best understood from the following detailed description when read with the accompanying figures. It is emphasized that, in accordance with the standard practice in the industry, various features are not necessarily drawn to scale, and are used for illustration purposes only. Where a scale is shown, explicitly or implicitly, it provides only one illustrative example. In other embodiments, the dimensions of the various features may be arbitrarily increased or reduced for clarity of discussion.

For a fuller understanding of the nature and advantages of the present invention, reference is made to the following detailed description of preferred embodiments and in connection with the accompanying drawings, in which:

FIG. 1 shows an exemplary biopotential measurement system, in accordance with some embodiments of the disclosure provided herein;

FIG. 2 shows an exemplary pace pulse, in accordance with some embodiments of the disclosure provided herein;

FIG. 3 is an exemplary timing diagram of the arrival of a pace pulse, in accordance with some embodiments of the disclosure provided herein;

FIG. 4 is an exemplary timing diagram of the arrival of a pace pulse, in accordance with some embodiments of the disclosure provided herein;

FIG. 5 is an exemplary histogram of the pace pulse detectability, in accordance with some embodiments of the disclosure provided herein;

FIG. 6 is an exemplary diagram depicting the generation of ECG signals both with pace detection and without thereof, in accordance with some embodiments of the disclosure provided herein;

FIG. 7 is an exemplary diagram of a variety of pace data taken different sampling rates, in accordance with some embodiments of the disclosure provided herein;

FIG. 8 is an exemplary figure showing changes in capacitor charges during impedance changes and sampling effects thereof, in accordance with some embodiments of the disclosure provided herein;

FIG. 9 is an exemplary figure demonstrating ECG amplitudes as a function of data rates, in accordance with some embodiments of the disclosure provided herein;

FIG. 10 is an exemplary ECG sampling circuit, in accordance with some embodiments of the disclosure provided herein;

FIG. 11 is an exemplary schematic demonstrative of the compatibility of ECG measurements with other measurements, in accordance with some embodiments of the disclosure provided herein; and,

FIG. 12 shows an exemplary power comparison table, in accordance with some embodiments of the disclosure provided herein.

### DETAILED DESCRIPTION

The present disclosure relates to medical equipment. The present disclosure relates to medical equipment. More specifically, this disclosure describes apparatuses and systems for pace detection while measuring biopotentials. And, in particular, the present disclosure relates to a biopotential measure device which isolates pace pulses from the measured ECG signal to provide a true differential biopotential measurement.

The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Other objects, advantages and novel features of the disclosure are set forth in the proceeding in view of the drawings where applicable.

In the field of biological electrical measurements, it has been the general practice to employ conventional ECG solutions which use dedicated circuit monitoring for slope of pacemaker signals while attempting to ignore ECG signals. This is due to the high bandwidths of the pace pulses having rising times of less than 100 μs.

Those concerned with the development of biopotential amplifiers have long recognized the need for isolating the measurement subject from the pace pulses originating from the pacemaker. There has also been a continuous need for amplifier devices which are immune to extraneous common mode voltages and the interference of EMI and ground loop currents.

One of the most critical problems confronting designers of biopotential amplifiers has been to electrically isolate the measurement subject. In prior art measurement apparatus, the measurement subject is connected to the measuring circuit ground by a third electrode which places the subject into a position of coming in contact with another electrical system whereby extraneous currents and voltages may be developed. This present disclose contemplates solutions thereof.

The general purpose of the present disclosure is to provide a biopotential measurement system and circuit which embraces all the advantages of amplifiers employed in the state of the art which possesses none of the aforementioned disadvantages. To attain this, the present disclosure attains this through a novel current detection method, rather a voltage detection. However, the use of voltage detection amplifiers is not beyond the scope of the present invention.

Biopotential measurement is can be used in modern medical procedures. For example, biopotentials can be used for electrocardiogram (ECG), electroencephalogram (EEG), electromyography (EMG), etc.

An example of an electrical measurement circuit connected to electrodes can include a high impedance node (>>10 MΩ) that facilitates the measurement of the potential at the electrodes. These potential measurements fall in two categories: capacitive pick-up with insulated electrode with no direct current (DC) path from the electrode to the measurement circuit or a contact electrode with resistive connection. Most of the practical measurements of biopotentials are with contact electrodes.

A “lead-off” detect measurement is carried out by arranging a pull up resistors to the input and the other side connected to the ground or power supply. These resistors establish a set of potentials at the input which are “disturbed” or changed when the leads or open or there is a path for current to flow thru the body. The change in the equilibrium potential at the input are measured by comparing the case of open leads to a situation where leads are attached to the body. This will be discussed in greater detail later in the disclosure.

A high lead impedance typically resulting from use of dry electrodes makes this measurement either difficult or unreliable. This is because high impedance of the lead makes the changes in the potential at the input very small and thus hard to measure.

The impedance of the pull-up resistor acts to divide the biopotential signal as measured by the circuit. A rough estimate for the measured potential compared to the “true potential” is given by a simple formula:

$${v}_{m} = {v}_{e l} ( \frac{{R}_{pull}}{{R}_{pull} + {R}_{e l}} )$$

where, vₘ is the measured voltage, vₑₗ is the electrode voltage, Rₚᵤₗₗ is the pull-up resistance and Rei is the electrode resistance.

Considering the case for ECG which produces some of the largest signals. The peak of the ECG is ˜1-2 mV. In a case where Rₑₗ˜10Rₚᵤₗₗ, the measured signal will be 1/11^{th} the input and become very small.

The potential at the lead is a sum of two potentials: internal potentials developed by the biological function and induced potential due to induction and polarization by the external magnetic and electric fields i.e., vₑₗ=v_{b}+v_{ind}. Many techniques have been suggested to separate or suppress the induced potentials. The present disclosure contemplates a novel system and apparatus which promises to be superior to the previously suggested techniques.

Some of the objects of the present disclosure comprise the following:
Pace detection works and is very robust with detection probabilities >95% and perhaps close to 100%.
Works no matter where the pace pulse appears on the ECG waveform.
Meets pace detect criteria and call out when pace pulse occurred.
The pace detect filter is quite simple and easily implemented on chip to reduce data transfer rates.
Pace-free ECG can also be generated on chip.
Sensing cap on-chip, with next-gen Coolidge accompanied by other improvements, allows to meet all the requirements of ECG including CMRR.

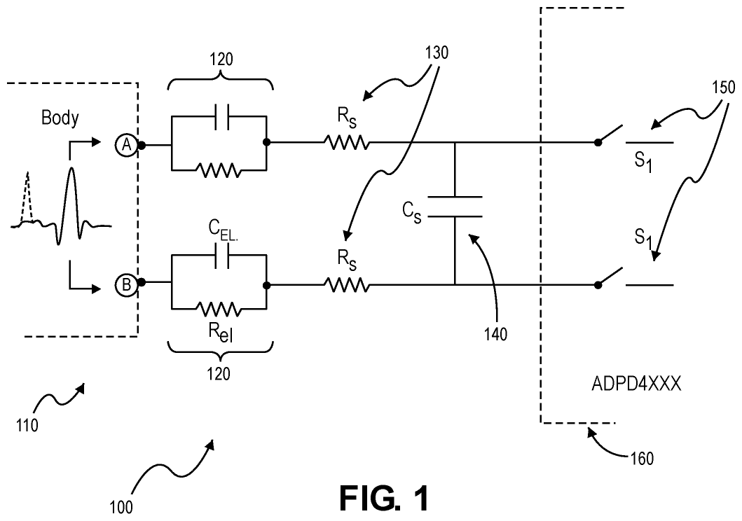

FIG. 1 shows an exemplary biopotential measurement system 100, in accordance with some embodiments of the disclosure provided herein. Biopotential measurement system 100 comprises body interface 110, electrodes 120, sense resistors 130, sampling capacitor 140, circuit interface 160, and switches 150

In practice, the bio-potentials are sampled with floating capacitor with relatively long time constant, which are then sampled rapidly by closing switches S1. Turning to FIG. 1, the present disclosure will now go through an exemplary ECG and pace measurement method.

The proceeding is a cursory summary describing how this method of measuring ECG works. The sampling circuit consists of two series resistors Rₛ 130 and a sampling cap Cₛ 140. The electrodes 120 themselves have some electrode resistance and capacitance. The real-world electrode model may be more complex but this model will sufficient without loss of generality for understanding the principle.

Points A and B on the body have different potential due to moving dipole originating from the currents in the heart. This potential difference V_{AB} charges sense capacitor Cₛ with time constant:

τ=(2Rₛ+2Rₑₗ)Cₛ (1)

The capacitor charges for a few time constants so that the potential across the capacitor V_{c} is very close to V_{AB}. In fact,

$${V}_{c} = {V}_{A B} ( 1 - {e}^{- \frac{t}{τ}} ) \qquad ( 2 )$$

Once the capacitor is charged close to V_{AB}, we quickly discharge the capacitor by closing switch S₁ into a charge measuring circuit and measure charge:

Qₛ=CₛV_{c} (3)

The discharge time τ_{disch}<<τ. This returns the capacitor to zero potential difference. At which point switch opens and the cycle repeats.

For ECG measurement at 300 Hz or sampling time tₛ of ˜333 ms, so we may set τ consistent with such a measurement such that

$$τ \leq ( \frac{{t}_{s}}{3} )$$

to faithfully represent the body potential V_{AB} according to Equation 2.

The same circuit measures pace pulses with high fidelity and extremely low error rate.

We note that the pace pulse is just like measurement of V_{AB} except that:

1. It has higher BW than ECG pulse.

2. It can arrive at any time during the sampling window tₛ.

3. It can vary from 100 μs to a few ms in width and a few mV to 100's of mV in amplitude—see text box below.

4. It is “DC balanced” as shown in the diagram. The “recharge” pulse is at least 10× longer in time than the pace pulse.

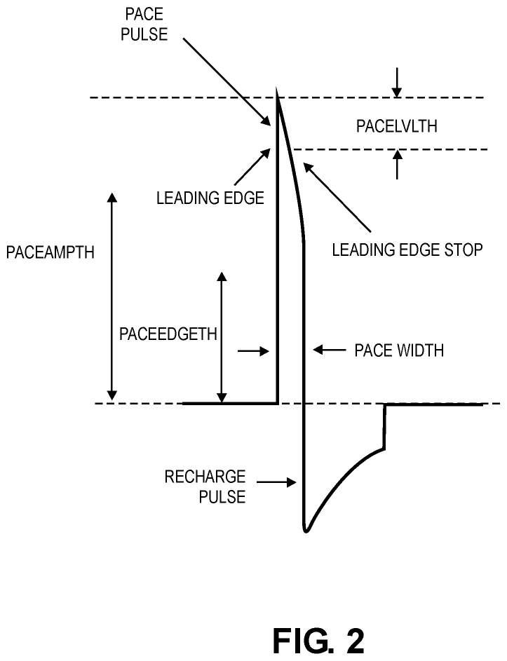

FIG. 2 shows an exemplary pace pulse, in accordance with some embodiments of the disclosure provided herein. It can be observed that the pace pulse drops after the impulse of the leading edge. and resultant pulse width parameter. Additionally, one of ordinary skill in the art recognizes the recharge time.

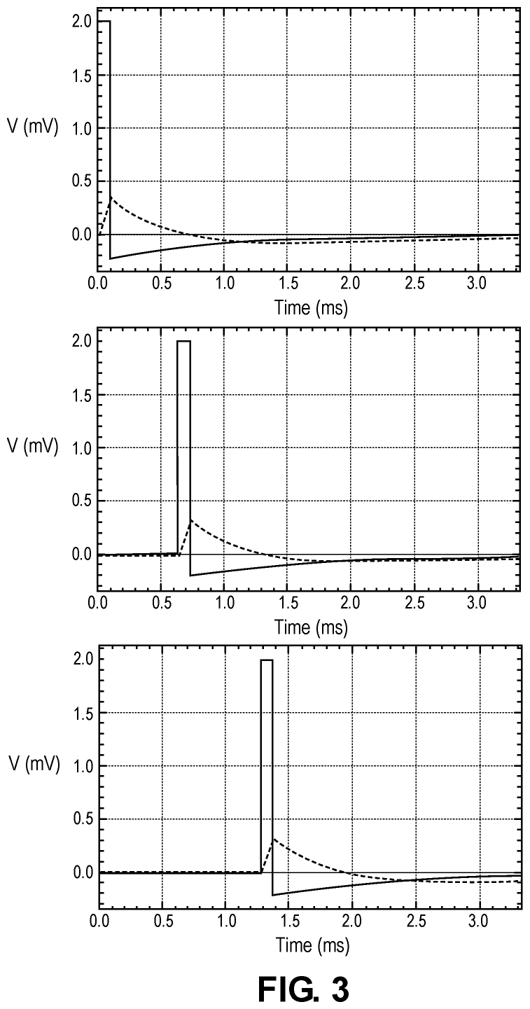

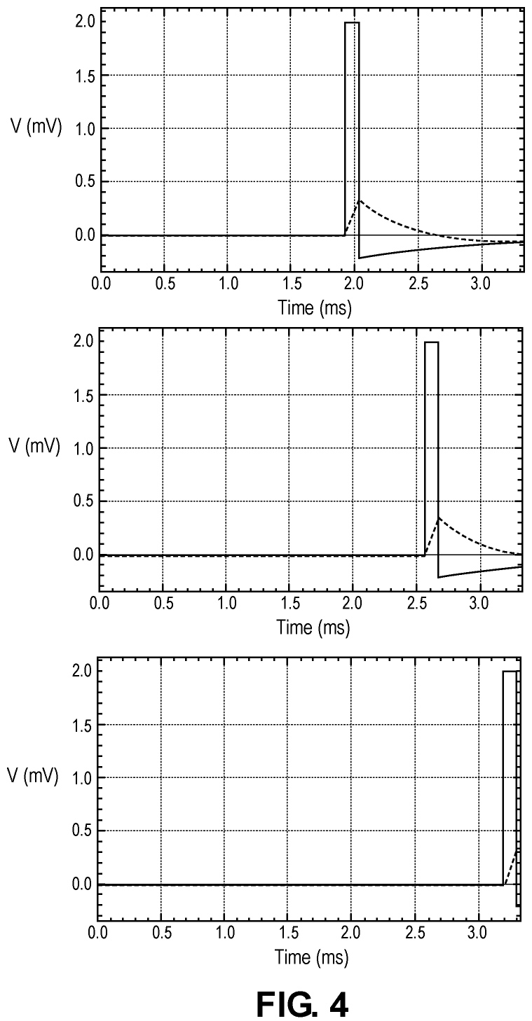

In order to understand detection of pace pulses using the circuit shown above, we simply need to remember that the capacitor voltage always tries to follow the voltage V_{AB}. Thus, as soon as the pace pulse is completed, the capacitor begins to discharge thru the body unless the voltage/charge on the sense capacitor is read-out before the pace pulse induced charge is lost thru the body. In the series of pictures depicted in FIGS. 3-4, we illustrate this by showing the voltage across the sense capacitor over time and the voltage just before the closure of the switch.

FIGS. 3-4 is an exemplary timing diagram of the arrival of a pace pulse, in accordance with some embodiments of the disclosure provided herein. FIGS. 3-4 depicts the arrival of pace pulse and voltage on the sense capacitor. Pace pulse of 2 mV height and 100 μs duration in Blue and voltage on the sense capacitor in Gold for 300 Hz sampling rate or total sampling tome 3.33 ms. Rₛ=500 kΩ, Rₑₗ=50 kΩ, and Cₛ=470 pF. The pace pulse arrives at any point between the samples and thus depending on the timing, there may not be any charge left on the capacitor from the pace pulse.

The probability that a pace pulse is detected—assuming random arrival time within the sampling window—can be constructed. Since the pace pulse only exists for a few samples taken at high sample rate, rapid changes in the sampled signal can be found by using difference operation or a median filter. There are many other strategies. In the example below, a simple 3 point (double-sided) median filter is used.

pace=S−Median[S,1] (4)

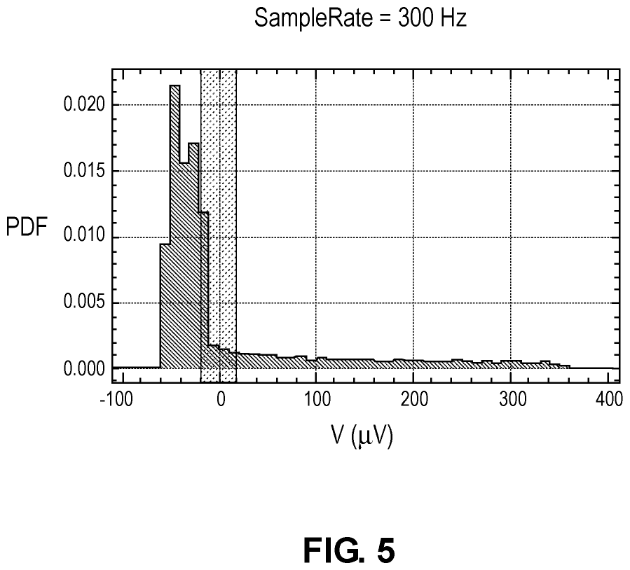

FIG. 5 is an exemplary histogram of the pace pulse detectability, in accordance with some embodiments of the disclosure provided herein. FIG. 5 is a histogram of the pace pulse detectability. The red region is voltage noise of the measurement system. The bars are drawn at ±3vₙ.

In the above distribution of FIG. 5, we can estimate the probability of catching the pace pulse. That is all the area under the curve except for the red shaded region. This is around 0.9 but as we shall see below, this simple algorithm works on the actual data with success rate of ˜78%.

As the sample rate increases, there is not enough time for the capacitor to discharge thru the body after the pace pulse no matter when it arrives. Thus, the probability of finding the pace pulse increases.

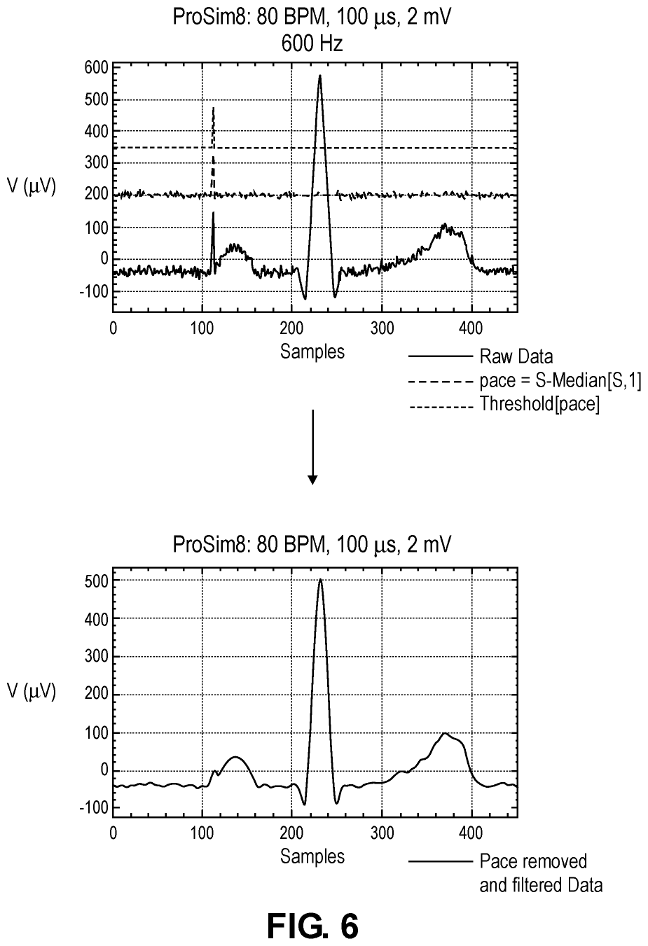

FIG. 6 is an exemplary diagram depicting the generation of ECG signals both with pace detection and without thereof, in accordance with some embodiments of the disclosure provided herein. This figure shows the example of the above with real world data at 600 Hz.

FIG. 6 exemplifies detecting pace and generating both pace marker as well as pace-free ECG. One can clearly see the spike on the data. Applying the above-mentioned filter to a long train of data—241 beats finds the pace pulse 78% of the time at 600 Hz with 100 μs pace width.

At 900 Hz, the same algorithm finds the pace pulse 90% of the time and at 1200 Hz this rises to 98% of the time. The few cases of not finding the pace is triggering from noise spikes and I rejected detection if two events were discovered in a single window. Thus, a small tweak will bring these numbers close to 100%

If the pace width is increased to 200 μs, this simple algorithm finds the pace pulse 87% of the time even at 600 Hz and rises to 98-100% at higher sample rates.

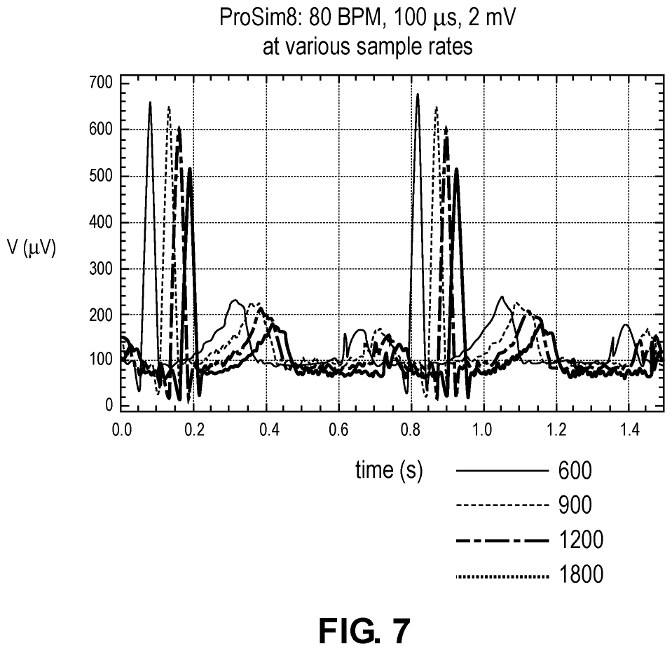

FIG. 7 is an exemplary diagram of a variety of pace data taken different sampling rates, in accordance with some embodiments of the disclosure provided herein. ECG amplitudes in the context of sample rates will now be discussed in greater detail.

One of ordinary skill in the art can appreciate that the probability of pace detection increases as the data rate increases. But the ECG will not the charge sense capacitor fully according to Equation 2. This can be seen in the data in FIG. 7.

One can clearly see the decrease in the sampled amplitude at higher sample rate consistent with equation 2. Therefore, it can be compensated by using equation 2. This requires measurement of τ. But the requirements on measurement of τ are not very high. For example, for clinical customers with wet electrodes, the electrode impedance will change from say 50-200 kΩ while we may set Rₛ=500 kΩ. We can compensate for the sample rate using equation 2 by using nominal values.

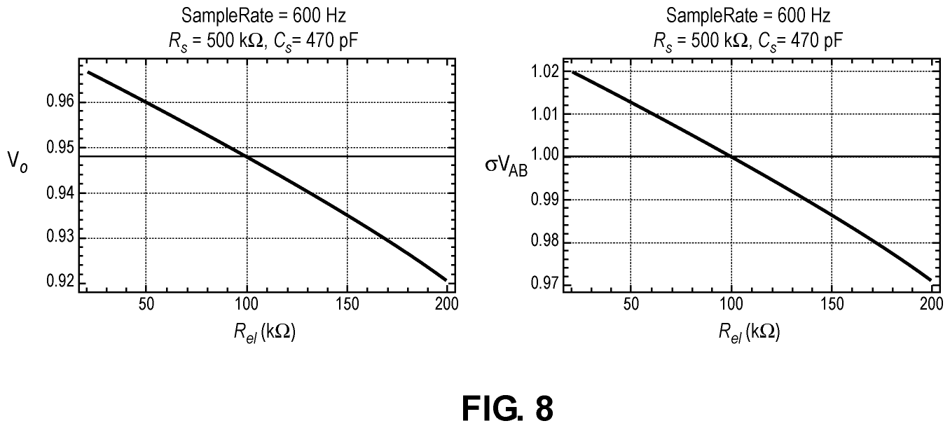

FIG. 8 is an exemplary figure showing changes in capacitor charges during impedance changes and sampling effects thereof, in accordance with some embodiments of the disclosure provided herein. As electrode impedance changes, the capacitor no longer charges to the same extent. The measured V_{c} relative to the actual potential is shown in the left panel. The relative change—assuming it is 100 kΩ is shown in the right panel.

The graph in FIG. 8 shows that the true ECG amplitude can be extracted without knowing the electrode impedance with very high accuracy. In the case of ProSim measurement, we knew τ, and so we can compensate for the change in ECG amplitude in software.

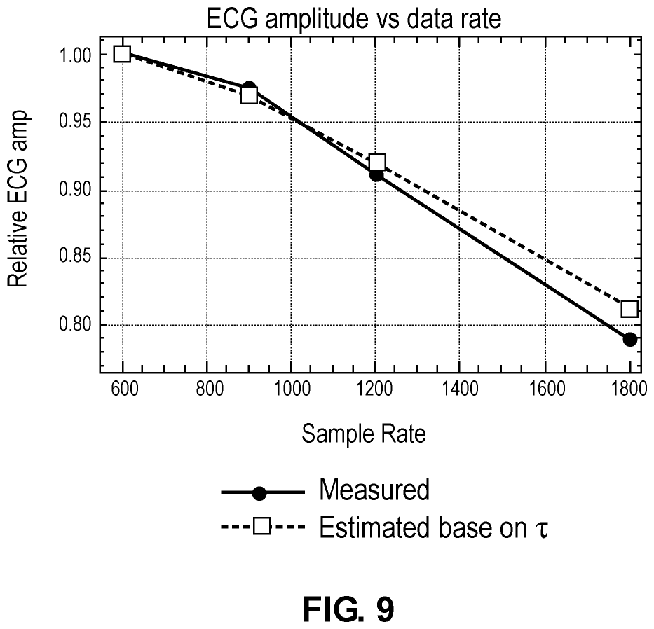

FIG. 9 is an exemplary figure demonstrating ECG amplitudes as a function of data rates, in accordance with some embodiments of the disclosure provided herein.

Note that the τ derived to fit the data is different than the estimated from the capacitor and resistor values due to varying different components.

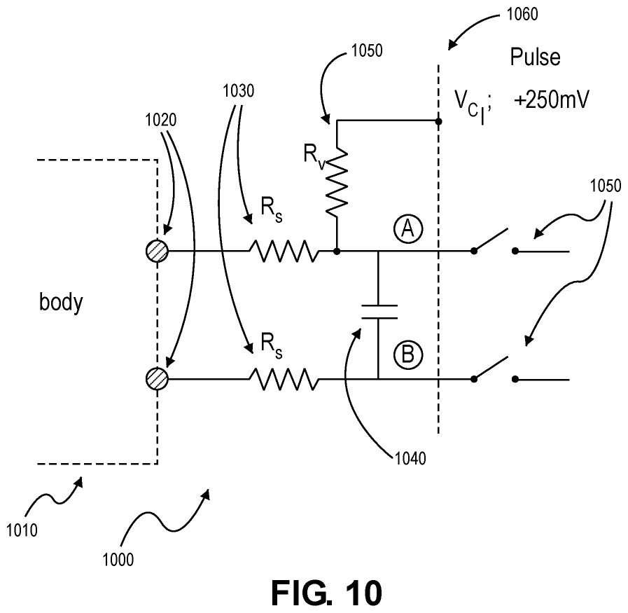

A new measurement of τ will now be discussed. FIG. 10 is an exemplary ECG sampling circuit 1000, in accordance with some embodiments of the disclosure provided herein. ECG sampling circuit 1000 comprises body interface 1010, contacts 1020, sense resistors 1030, resistor 1050, capacitor 1040, circuit interface 1060 and switches 1050.

Consider tying node A in the circuit below to Vc1 pin. Now using a particular sequence of measurements, we can estimate r and hence electrode impedance. Here is a sequence of events associated with the circuit of FIG. 10.

Slot A: Measure ECG as one normally would in sleep-float mode. Vc1 is floating.

Slot B: Pulse Vc1 for a short time ˜1 μs (depending on the value of Rᵥ) to +250 mV. This will change the charge on the capacitor. Note that the two ends of the cap were left at ˜0.9 V at the end of Slot A. Node B in the circuit needs to be connected in a single ended fashion in order to apply 250 mV. Otherwise cap is still floating. This will charge the cap to a known potential. For example, Rᵥ=100 kΩ and Cₛ=470 pF will charge the cap to +5 mV with 1 μs Vc1 pulse. Another way to accomplish the same would be switch the cap to single ended, and use the pulse V_ref mode.

Slot C: Measure voltage on the cap by dumping cap with high input setting (Rᵢₙ=650011 and dump time of 1 μs. Let cap float again. Call this measurement M₁.

Slot D: Wait τ_{wait}˜10 μs and repeat the measurement as in Slot C. This will dump smaller charge as capacitor has discharges thru the body. some of the charge has been lost thru body. Call this measurement M₂.

Optional Slot E: Fully discharge the cap by shorting it and then floating it for next cycle of ECG measurement. This does not have to happen as it will happen thru body.

From these measurements, we can estimate the timescale τ and the electrode resistance in the ECG frequency band. It is easy to derive that:

$$\frac{{M}_{2}}{{M}_{1}} = {e}^{- \frac{{τ}_{wait}}{τ}} \qquad ( 5 )$$

Or,

$$τ = {τ}_{w a i t} log ( \frac{{M}_{1}}{{M}_{2}} ) \qquad ( 6 )$$

Note that we pick τ_{wait} such that we have good SNR to measure τ from Equation 5. Then from Equation 1, we can find electrode resistance.

We may be able to combine Slot B & C.

Also, these measurements can be made using impulse mode or running the sampling rate fast enough to see the effect of Slot B on measurements but running the Slot B at ½ the rate all the time. It can be ⅓^{rd} or ¼^{th} etc.

If M₂=M₁ then we have open thru the body and this represents lead-off.

Very large pace pulses will now be discussed. As the pace pulse becomes either wider or taller, more charge is transferred to the capacitor and becomes easy to detect. Even a 200 mV, 2 ms pulse will not saturate the measurement and will generate pace signature that lasts a few samples. This requires an adjustment of the median filter that we used to identify the pulse. Since we are DC coupled, we recover instantaneously, and continue normal measurement of ECG as soon as pace pulse disappears, and capacitors are sampled and restored. We can clearly use software to “blank” the ECG measurement for 20-40 ms which is 18-36 samples at 900 Hz. Typical ECG front-ends are effectively AC coupled and hence it takes

$$\text{∼} \frac{1}{{f}_{h}}$$

time to recover where fₕ is the high pass corner for AC coupling.

Thus, our method is fundamentally robust to the previous methods. We do not require any high-speed sampling and as we shall see below, any improvement in amplifiers and dynamic range can be used to further reduce the power.

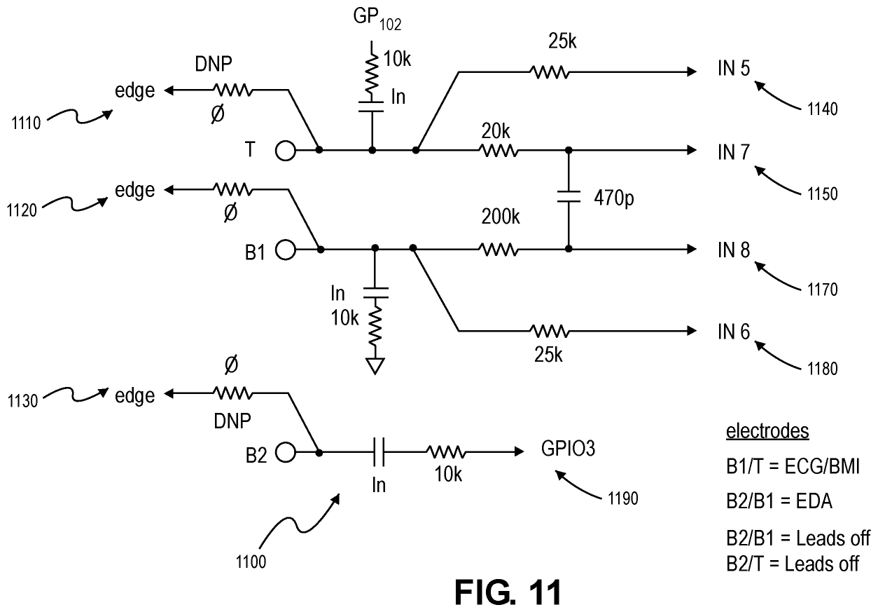

FIG. 11 is an exemplary schematic 1100 demonstrative of the compatibility of ECG measurements with other measurements 1190, in accordance with some embodiments of the disclosure provided herein. ECG measurement comprise edge leads 1110, 1120, 1130 and inputs 1140, 1150, 1170, and 1180.

Compatibility with measurement of other parameters is now described. As the diagram below shows, we can easily combine ECG measurements with other measurements. FIG. 11 is for impedance and ECG measurements. A few more timeslots can be added for photoplethysmography (PPG), etc.

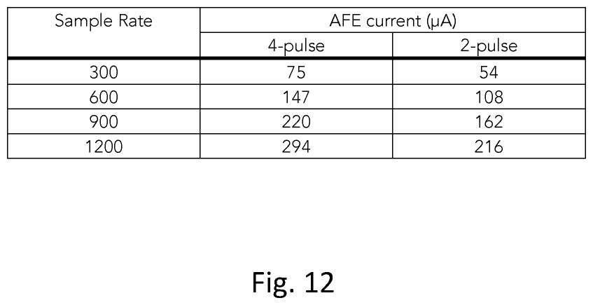

FIG. 12 shows an exemplary power comparison table, in accordance with some embodiments of the disclosure provided herein. Power effects will now be discussed. The faster sampling rate increases the power consumed by a circuit, e.g., ADPD4100. The power numbers vs sample rate for ECG alone are shown in FIG. 12.

In the current version of Coolidge, we use 4-pulse sequence to dump charge on the detector. By doubling the dynamic range of the integrator stage (start from −Qₘₐₓ rather than 0) we can reduce the pulses and thus save on power.

Noise is simply amplifier noise imposed on the sense capacitor. This noise is independent and occurs every time the capacitor is read. Our filtered (DC to 40 Hz) noise is ˜3.5 μV rms when sample rate is 300 Hz.

The following describes how noise can be reduces by factor of 3 to 4 for the same power:

1. Double the dynamic range of integrator and then with R_{f}=100 k, choose Rᵢₙₜ=200 k. This means that input referred noise will be lower by just less than factor of 2.

2. Reduce TIA noise from present effective input noise density of ˜20-25 nV/rtHz to less than 10 nV/rtHz. This will decrease the size of the cap for the same noise and allow us to bring it on board and further reduce EMI, etc. Lowering cap also increases the effective input impedance. More aggressively power cycle amplifiers/newer more efficient amps.

3. It seems that we can easily imagine 2×-3× improvement in noise. This will take it to 1-1.5 μV rms or 6-9 μVₚₚ making it as good as the best.

Note that this improvement automatically translates to meeting diagnostic requirements as well as EEG measurements!

Common mode rejection ration (CMRR) will now be discussed.

CMRR can be improved by moving the sampling system on chip or by disconnecting the cap from the world while dumping its charge. In my simple model, since the CM drive is attached to the two inputs of the differential amplifier by slightly different impedance paths—unbalanced input impedance of the amplifier, mismatched electrodes etc.—or small differences in voltage at the two pins will cause differential current to flow during the dump time.

All these effects can be eliminated by disconnecting sense cap from the rest of the world before dumping. This always keeps the cap floating and there is no “push and pull” of the inputs by the CM drive.

### SELECT EXAMPLES

Example 1 provides a method for measuring pace pulses using a sense capacitor and altering the sampling rate comprising receiving a first biopotential signal, receiving a second biopotential signal, passing the first biopotential signal through a first series resistor, passing the second biopotential signal through a second series resistor, processing the difference between the first and second signal with a capacitor disposed there-between, and sampling the processed difference between the first and second signal at a first sampling rate.

Example 2 provides a method for measuring pace pulses using a sense capacitor and altering the sampling rate of example 1 further comprising measuring the RC time constant of the processed difference between the first and second signal.

Example 3 provides a method for measuring pace pulses using a sense capacitor and altering the sampling rate of example 2 further comprising allowing the capacitor to charge before sampling.

Example 4 provides a method for measuring pace pulses using a sense capacitor and altering the sampling rate of example 3 wherein the temporal allowance is two or more RC time constants.

Example 5 provides a method for measuring pace pulses using a sense capacitor and altering the sampling rate of example 4 whereby the two or more RC time constants allows the processed difference between the first and second signal to approach the difference between the first and second biopotential signal.

Example 6 provides a method for measuring pace pulses using a sense capacitor and altering the sampling rate of example 5 further comprising adjusting the sampling rate to meet this criterion.

Example 7 provides a method for measuring pace pulses using a sense capacitor and altering the sampling rate of example 1 further comprising adjust the sampling rate so that it is low enough to ensure that the capacitor discharges before the next sample yet fast enough to yield a sample window which captures a pace pulse event.

Example 8 provides a method for measuring pace pulses using a sense capacitor and altering the sampling rate of example 1 further comprising constructing a probability table to detect the pace pulse assuming random arrival time within a sampling window.

Example 9 provides a method for measuring pace pulses using a sense capacitor and altering the sampling rate of example 8 further comprising analyzing the sampled signal for rapid changes.

Example 10 provides a method for measuring pace pulses using a sense capacitor and altering the sampling rate of example 8 wherein the analysis is performed using a difference operation.

Example 11 provides a method for measuring pace pulses using a sense capacitor and altering the sampling rate of example 8 wherein the analysis is performed using a filter.

Example 12 provides a method for measuring pace pulses using a sense capacitor and altering the sampling rate of example 11 wherein the filter is a median filter.

Example 13 provides a method for measuring pace pulses using a sense capacitor and altering the sampling rate of example 11 wherein the analysis comprises analyzing the filtered data by looking for a spike.

Example 14 provides a method for measuring pace pulses using a sense capacitor and altering the sampling rate of example 13 wherein the spike analysis is performed in the time domain.

Example 15 provides a method for measuring pace pulses using a sense capacitor and altering the sampling rate of example 13 wherein the spike analysis is performed in the frequency domain.

Example 16 provides a method for measuring pace pulses using a sense capacitor and altering the sampling rate of example 14 wherein the spike analysis uses a threshold to identify a pulse.

Example 17 provides a method for measuring pace pulses using a sense capacitor and altering the sampling rate of example 11 further comprising identifying the pace pulse in the sampled signal.

Example 18 provides a method for measuring pace pulses using a sense capacitor and altering the sampling rate of example 13 further comprising identifying an ECG signal in the sampled signal.

Example 19 provides a method for measuring pace pulses using a sense capacitor and altering the sampling rate of example 11 further comprising removing the pace pulse from the sampled signal.

Example 20 provides an apparatus for measuring pace pulses using a sense capacitor and altering the sampling rate comprising a first electrode having a parasitic resistance and capacitance configured to receive a first biopotential signal, a second electrode having a parasitic resistance and capacitance configured to receive a second biopotential signal, a first series resistor having a first and second terminal, the first terminal connected to the first electrode, a second series resistor having a first and second terminal, the first terminal connected to the second electrode, a sense capacitor wired between the second terminals of the first and second series resistors, a first switch in electrical communication with the second terminal of the first series resistors, a second switch in electrical communication with the second terminal of the second series resistors, and a switching controller configured to sample at a frequency low enough to allow the sense capacitor to charge to a potential approaching a difference measured between the first and second electrode.

Having thus described several aspects and embodiments of the technology of this application, it is to be appreciated that various alterations, modifications, and improvements will readily occur to those of ordinary skill in the art. Such alterations, modifications, and improvements are intended to be within the spirit and scope of the technology described in the application. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein.

Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The foregoing outlines features of one or more embodiments of the subject matter disclosed herein. These embodiments are provided to enable a person having ordinary skill in the art (PHOSITA) to better understand various aspects of the present disclosure. Certain well-understood terms, as well as underlying technologies and/or standards may be referenced without being described in detail. It is anticipated that the PHOSITA will possess or have access to background knowledge or information in those technologies and standards sufficient to practice the teachings of the present disclosure.

The PHOSITA will appreciate that they may readily use the present disclosure as a basis for designing or modifying other processes, structures, or variations for carrying out the same purposes and/or achieving the same advantages of the embodiments introduced herein. The PHOSITA will also recognize that such equivalent constructions do not depart from the spirit and scope of the present disclosure, and that they may make various changes, substitutions, and alterations herein without departing from the spirit and scope of the present disclosure.

The above-described embodiments may be implemented in any of numerous ways. One or more aspects and embodiments of the present application involving the performance of processes or methods may utilize program instructions executable by a device (e.g., a computer, a processor, or other device) to perform, or control performance of, the processes or methods.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above.

The computer readable medium or media may be transportable, such that the program or programs stored thereon may be loaded onto one or more different computers or other processors to implement various ones of the aspects described above. In some embodiments, computer readable media may be non-transitory media.

Note that the activities discussed above with reference to the FIGURES which are applicable to any integrated circuit that involves signal processing (for example, gesture signal processing, video signal processing, audio signal processing, analog-to-digital conversion, digital-to-analog conversion), particularly those that can execute specialized software programs or algorithms, some of which may be associated with processing digitized real-time data.

In some cases, the teachings of the present disclosure may be encoded into one or more tangible, non-transitory computer-readable mediums having stored thereon executable instructions that, when executed, instruct a programmable device (such as a processor or DSP) to perform the methods or functions disclosed herein. In cases where the teachings herein are embodied at least partly in a hardware device (such as an ASIC, IP block, or SoC), a non-transitory medium could include a hardware device hardware-programmed with logic to perform the methods or functions disclosed herein. The teachings could also be practiced in the form of Register Transfer Level (RTL) or other hardware description language such as VHDL or Verilog, which can be used to program a fabrication process to produce the hardware elements disclosed.

In example implementations, at least some portions of the processing activities outlined herein may also be implemented in software. In some embodiments, one or more of these features may be implemented in hardware provided external to the elements of the disclosed figures, or consolidated in any appropriate manner to achieve the intended functionality. The various components may include software (or reciprocating software) that can coordinate in order to achieve the operations as outlined herein. In still other embodiments, these elements may include any suitable algorithms, hardware, software, components, modules, interfaces, or objects that facilitate the operations thereof.

Any suitably-configured processor component can execute any type of instructions associated with the data to achieve the operations detailed herein. Any processor disclosed herein could transform an element or an article (for example, data) from one state or thing to another state or thing. In another example, some activities outlined herein may be implemented with fixed logic or programmable logic (for example, software and/or computer instructions executed by a processor) and the elements identified herein could be some type of a programmable processor, programmable digital logic (for example, an FPGA, an erasable programmable read only memory (EPROM), an electrically erasable programmable read only memory (EEPROM)), an ASIC that includes digital logic, software, code, electronic instructions, flash memory, optical disks, CD-ROMs, DVD ROMs, magnetic or optical cards, other types of machine-readable mediums suitable for storing electronic instructions, or any suitable combination thereof.

In operation, processors may store information in any suitable type of non-transitory storage medium (for example, random access memory (RAM), read only memory (ROM), FPGA, EPROM, electrically erasable programmable ROM (EEPROM), etc.), software, hardware, or in any other suitable component, device, element, or object where appropriate and based on particular needs. Further, the information being tracked, sent, received, or stored in a processor could be provided in any database, register, table, cache, queue, control list, or storage structure, based on particular needs and implementations, all of which could be referenced in any suitable timeframe.

Any of the memory items discussed herein should be construed as being encompassed within the broad term ‘memory.’ Similarly, any of the potential processing elements, modules, and machines described herein should be construed as being encompassed within the broad term ‘microprocessor’ or ‘processor.’ Furthermore, in various embodiments, the processors, memories, network cards, buses, storage devices, related peripherals, and other hardware elements described herein may be realized by a processor, memory, and other related devices configured by software or firmware to emulate or virtualize the functions of those hardware elements.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a personal digital assistant (PDA), a smart phone, a mobile phone, an iPad, or any other suitable portable or fixed electronic device.

Also, a computer may have one or more input and output devices. These devices can be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks or wired networks.

Computer-executable instructions may be in many forms, such as program modules, executed by one or more computers or other devices. Generally, program modules include routines, programs, objects, components, data structures, etc. that performs particular tasks or implement particular abstract data types. Typically, the functionality of the program modules may be combined or distributed as desired in various embodiments.

The terms “program” or “software” are used herein in a generic sense to refer to any type of computer code or set of computer-executable instructions that may be employed to program a computer or other processor to implement various aspects as described above. Additionally, it should be appreciated that according to one aspect, one or more computer programs that when executed perform methods of the present application need not reside on a single computer or processor, but may be distributed in a modular fashion among a number of different computers or processors to implement various aspects of the present application.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code may be executed on any suitable processor or collection of processors, whether provided in a single computer or distributed among multiple computers.

Computer program logic implementing all or part of the functionality described herein is embodied in various forms, including, but in no way limited to, a source code form, a computer executable form, a hardware description form, and various intermediate forms (for example, mask works, or forms generated by an assembler, compiler, linker, or locator). In an example, source code includes a series of computer program instructions implemented in various programming languages, such as an object code, an assembly language, or a high-level language such as OpenCL, RTL, Verilog, VHDL, Fortran, C, C++, JAVA, or HTML for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

In some embodiments, any number of electrical circuits of the FIGURES may be implemented on a board of an associated electronic device. The board can be a general circuit board that can hold various components of the internal electronic system of the electronic device and, further, provide connectors for other peripherals. More specifically, the board can provide the electrical connections by which the other components of the system can communicate electrically. Any suitable processors (inclusive of digital signal processors, microprocessors, supporting chipsets, etc.), memory elements, etc. can be suitably coupled to the board based on particular configuration needs, processing demands, computer designs, etc.

Other components such as external storage, additional sensors, controllers for audio/video display, and peripheral devices may be attached to the board as plug-in cards, via cables, or integrated into the board itself. In another example embodiment, the electrical circuits of the FIGURES may be implemented as standalone modules (e.g., a device with associated components and circuitry configured to perform a specific application or function) or implemented as plug-in modules into application-specific hardware of electronic devices.

Note that with the numerous examples provided herein, interaction may be described in terms of two, three, four, or more electrical components. However, this has been done for purposes of clarity and example only. It should be appreciated that the system can be consolidated in any suitable manner. Along similar design alternatives, any of the illustrated components, modules, and elements of the FIGURES may be combined in various possible configurations, all of which are clearly within the broad scope of this disclosure.

In certain cases, it may be easier to describe one or more of the functionalities of a given set of flows by only referencing a limited number of electrical elements. It should be appreciated that the electrical circuits of the FIGURES and its teachings are readily scalable and can accommodate a large number of components, as well as more complicated/sophisticated arrangements and configurations. Accordingly, the examples provided should not limit the scope or inhibit the broad teachings of the electrical circuits as potentially applied to a myriad of other architectures.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

### Interpretation of Terms

All definitions, as defined and used herein, should be understood to control over dictionary definitions, definitions in documents incorporated by reference, and/or ordinary meanings of the defined terms. Unless the context clearly requires otherwise, throughout the description and the claims:

“comprise,” “comprising,” and the like are to be construed in an inclusive sense, as opposed to an exclusive or exhaustive sense; that is to say, in the sense of “including, but not limited to”.

“connected,” “coupled,” or any variant thereof, means any connection or coupling, either direct or indirect, between two or more elements; the coupling or connection between the elements can be physical, logical, or a combination thereof.

“herein,” “above,” “below,” and words of similar import, when used to describe this specification shall refer to this specification as a whole and not to any particular portions of this specification.

“or,” in reference to a list of two or more items, covers all of the following interpretations of the word: any of the items in the list, all of the items in the list, and any combination of the items in the list.

the singular forms “a”, “an” and “the” also include the meaning of any appropriate plural forms.

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present) depend on the specific orientation of the apparatus described and illustrated. The subject matter described herein may assume various alternative orientations. Accordingly, these directional terms are not strictly defined and should not be interpreted narrowly.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined.

Elements other than those specifically identified by the “and/or” clause may optionally be present, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “between” is to be inclusive unless indicated otherwise. For example, “between A and B” includes A and B unless indicated otherwise.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Numerous other changes, substitutions, variations, alterations, and modifications may be ascertained to one skilled in the art and it is intended that the present disclosure encompass all such changes, substitutions, variations, alterations, and modifications as falling within the scope of the appended claims.

In order to assist the United States Patent and Trademark Office (USPTO) and, additionally, any readers of any patent issued on this application in interpreting the claims appended hereto, Applicant wishes to note that the Applicant: (a) does not intend any of the appended claims to invoke 35 U.S.C. § 112(f) as it exists on the date of the filing hereof unless the words “means for” or “steps for” are specifically used in the particular claims; and (b) does not intend, by any statement in the disclosure, to limit this disclosure in any way that is not otherwise reflected in the appended claims.

The present invention should therefore not be considered limited to the particular embodiments described above. Various modifications, equivalent processes, as well as numerous structures to which the present invention may be applicable, will be readily apparent to those skilled in the art to which the present invention is directed upon review of the present disclosure.

What is claimed is:

1. A method for measuring pace pulses using a sense capacitor and altering the sampling rate comprising:
receiving a first biopotential signal;
receiving a second biopotential signal;
passing the first biopotential signal through a first series resistor;
passing the second biopotential signal through a second series resistor;
processing the difference between the first and second signal with a capacitor disposed there-between;
sampling the processed difference between the first and second signal at a first sampling rate.

2. The method according to claim 1 further comprising measuring the RC time constant of the processed difference between the first and second signal.

3. The method according to claim 2 further comprising allowing the capacitor to charge before sampling.

4. The method according to claim 3 wherein the temporal allowance is two or more RC time constants.

5. The method according to claim 4 whereby the two or more RC time constants allows the processed difference between the first and second signal to approach the difference between the first and second biopotential signal.

6. The method according to claim 5 further comprising adjusting the sampling rate to meet this criterion.

7. The method according to claim 1 further comprising adjust the sampling rate so that it is low enough to ensure that the capacitor discharges before the next sample yet fast enough to yield a sample window which captures a pace pulse event.

8. The method according to claim 1 further comprising constructing a probability table to detect the pace pulse assuming random arrival time within a sampling window.

9. The method according to claim 8 further comprising analyzing the sampled signal for rapid changes.

10. The method according to claim 8 wherein the analysis is performed using a difference operation.

11. The method according to claim 8 wherein the analysis is performed using a filter.

12. The method according to claim 11 wherein the filter is a median filter.

13. The method according to claim 11 wherein the analysis comprises analyzing the filtered data by looking for a spike.

14. The method according to claim 13 wherein the spike analysis is performed in the time domain.

15. The method according to claim 13 wherein the spike analysis is performed in the frequency domain.

16. The method according to claim 14 wherein the spike analysis uses a threshold to identify a pulse.

17. The method according to claim 11 further comprising identifying the pace pulse in the sampled signal.

18. The method according to claim 13 further comprising identifying an ECG signal in the sampled signal.

19. The method according to claim 11 further comprising removing the pace pulse from the sampled signal.

20. An apparatus for measuring pace pulses using a sense capacitor and altering the sampling rate comprising:
a first electrode having a parasitic resistance and capacitance configured to receive a first biopotential signal;
a second electrode having a parasitic resistance and capacitance configured to receive a second biopotential signal;
a first series resistor having a first and second terminal, the first terminal connected to the first electrode;
a second series resistor having a first and second terminal, the first terminal connected to the second electrode;
a sense capacitor wired between the second terminals of the first and second series resistors;
a first switch in electrical communication with the second terminal of the first series resistors;
a second switch in electrical communication with the second terminal of the second series resistors; and
a switching controller configured to sample at a frequency low enough to allow the sense capacitor to charge to a potential approaching a difference measured between the first and second electrode.

21. An apparatus for measuring pace pulses using a sense capacitor and altering the sampling rate comprising:
means for receiving a first biopotential signal;
means for receiving a second biopotential signal;
means for passing the first biopotential signal through a first series resistor;
means for passing the second biopotential signal through a second series resistor;
means for processing the difference between the first and second signal with a capacitor disposed there-between;
means for sampling the processed difference between the first and second signal at a first sampling rate.
